(19) United States
(12) Patent Application Publication
(10) Pub. No.: US 2009/0227870 A1
(43) Pub. Date: Sep. 10, 2009

(54) METHOD OF DETERMINING A PARAMETER REPRESENTING AN ACOUSTIC PROPERTY OF A MATERIAL

(75) Inventors: Roy Gerardus Maria Kolkman, Lichtenvoorde (NL); Antonius Gerardus Johannes Maria van Leeuwen, Bussum (NL); Wiendelt Steenbergen, Enschede (NL)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: STICHTING VOOR FUNDAMENTEEL ONDERZOEK DER MATERIE, UTRECHT (NL)

(21) Appl. No.: 11/574,477

(22) PCT Filed: Aug. 31, 2004
(86) PCT No.: PCT/NL04/00603

§ 371 Date: Jun. 19, 2008

(51) Int. Cl. G01N 29/44; A61B 8/13; G01N 29/06

(52) U.S. Cl. 600/447; 600/442

(57) ABSTRACT

The invention relates to a method and system for determining a parameter representing an acoustic property of a material. The method comprises: generating an acoustic pressure wave in said material originating from a localized position; placing a plurality of acoustic receivers (6, 7) at mutually differing distances from the acoustic source (3); transforming a plurality of measured acoustic signals to represent field values in a common computational point; computing a signal representing a measure of overlap between said transformed plurality of acoustic signals as a function of said numerical estimates of said acoustic property parameter and deriving said acoustic property parameter from said overlap signal. Since the invention uses acoustic sources that are well localized in the material, the velocity calculations are simple and the geometry of the acoustic receivers in relation to the acoustic source can be exactly taken into account.

The invention relates to a method of determining a parameter representing an acoustic property of a material.

In U.S. Pat. No. 5,638,820 a method is illustrated using ultrasound for estimating the speed of sound in tissue. The system is based on placing a plurality of acoustic receivers at different positions in acoustic contact with the tissue; actuating an acoustic source to generate an acoustic pressure wave in said tissue; and measuring upon actuation of said acoustic source a plurality of acoustic signals from said acoustic receivers. The disclosure suggests varying an estimated acoustic velocity for obtaining increased image quality of the ultrasound images, using a spatial correlation function to define the image quality. The real speed is obtained from the variation of estimated speeds where the image quality is maximal. In the US patent, through means of a so called digital beam former acoustic waves that are recorded are correlated with acoustic sources in the tissue to visualize internal structures of the tissue. In case of high resolution imaging with a speed of sound deviating from the real speed of sound serious deformation artifacts will occur in the image.

The invention has as an object to provide an improved method for determining an acoustic property parameter.

In one aspect, this object is achieved by the features of claim 1. Specifically, according to the inventive method the acoustic sources are well localized and the near field geometry of the acoustic sensors is taken into account to provide a more reliable acoustic property parameter determination. In this aspect, the term “localized” implies that the acoustic waves are discernable as originating from a single “point” like volume, that is, a single volume that is localized in space. Accordingly, the inventive method comprises:

actuating an acoustic source to generate an acoustic pressure wave in said material originating from a localized source position;

placing an acoustic sensor comprising a plurality of acoustic receivers at mutually differing distances from the acoustic source in acoustic contact with the material;

measuring upon actuation of said acoustic source a plurality of acoustic signals from said acoustic receivers;

transforming said plurality of acoustic signals to represent field values in a common computational point, based on a numerical estimate of the acoustic velocity in material and the receiver positions relative to a localized position of the acoustic source;

computing a signal representing a measure of overlap between said transformed plurality of acoustic signals as a function of said numerical estimate of said acoustic property parameter; and

deriving said acoustic property parameter from said overlap signal.

According to the invention, a measure of overlap is directly derived from the spatially separated acoustic receivers giving a good spatial resolution of the acoustic signals. Furthermore, since the invention uses acoustic sources that are well localized in the material, the velocity calculations are simple and the geometry of the acoustic receivers in relation to the acoustic source can be exactly taken into account.

In another aspect the invention relates to a system according to the independent claim 14. Specifically, the system comprises an input section for inputting a plurality of acoustic signals derived from a plurality of acoustic receivers;

a computational unit for transforming said plurality of acoustic signals to represent field values in a common computational point, based on a numerical estimate of the acoustic velocity in material and the receiver positions relative to a localized position of the acoustic source; the computational unit further arranged for computing a signal representing a measure of overlap between said transformed plurality of acoustic signals as a function of said numerical estimate of said acoustic property parameter; and

an output section for outputting said acoustic property parameter derived from said overlap signal.

The system is arranged to cooperate with an acoustic source actuator to generate an acoustic pressure wave in material originating from a localized acoustic source position in the material and a sensor comprising a plurality of acoustic receivers to be placed in acoustic contact with the material at different positions thereon, for measuring, upon actuation of said acoustic source, a plurality of acoustic signals.

It is noted that such actuator/receiver arrangement is known from Roy G. M. Kolkman et al, In Vivo Photo acoustic imaging of blood vessels using an extreme-narrow aperture sensor, IEEE, Journal of selected topics in Quantum electronics, vol. 9, no. 2, pages 343-346, wherein a double ring acoustic sensor is disclosed having a narrow aperture, being able to perform acoustic imaging by scanning. This sensor is however used for imaging using a fixed acoustic sound velocity. In one preferred embodiment, the localized sources are provided by absorption of light pulses in the material. In another preferred embodiment, the sources are provided using an absorbing medium that is introduced in the material and that is responsive to the light pulses.

The invention will further be described with reference to the description, illustrating, by way of example only, further exemplary embodiments, features and benefits of the invention.

In the figures:

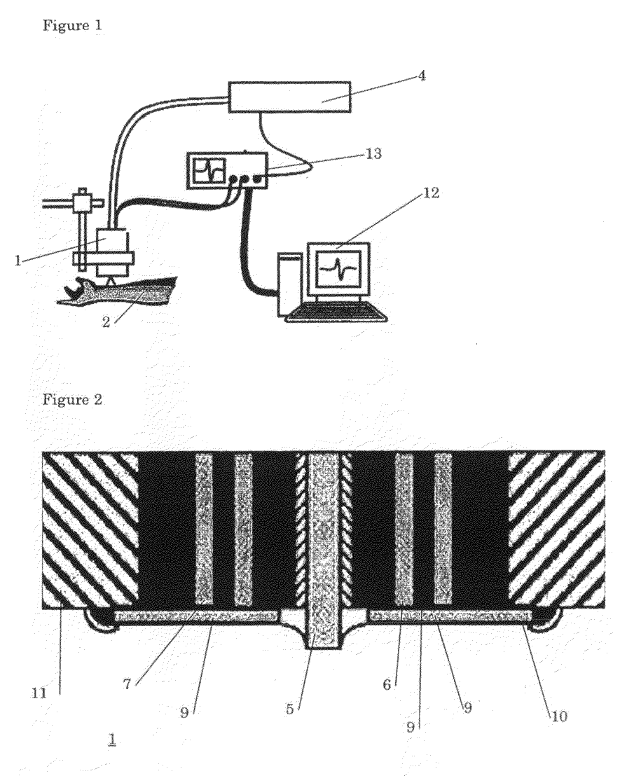

FIG. 1 shows an experimental setup for performing the method according to the invention; and

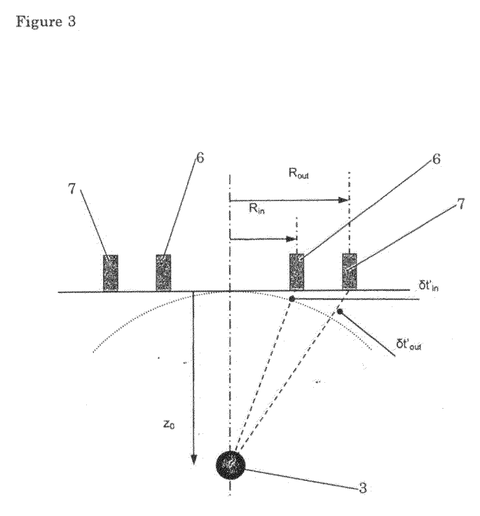

FIG. 2 shows a schematic cross sectional view of a preferred embodiment of the acoustic sensor according to the invention.

FIG. 3 shows schematically the geometry of the acoustic sensor when used according to the invention;

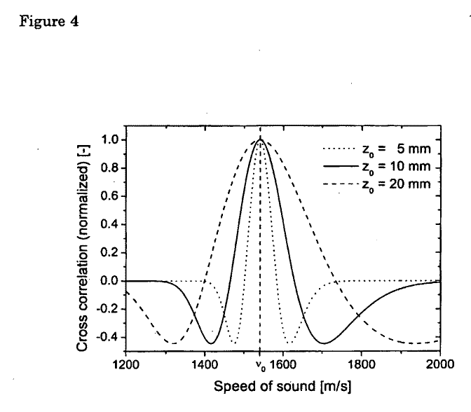

FIG. 4 shows a calculated correlation function for a source localized at various depths in tissue;

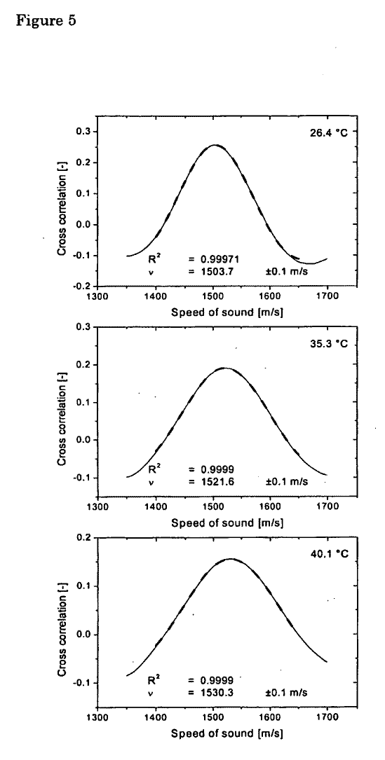

FIG. 5 shows a series of measured and calculated correlation functions for acoustic velocity measurements at different temperatures in water;

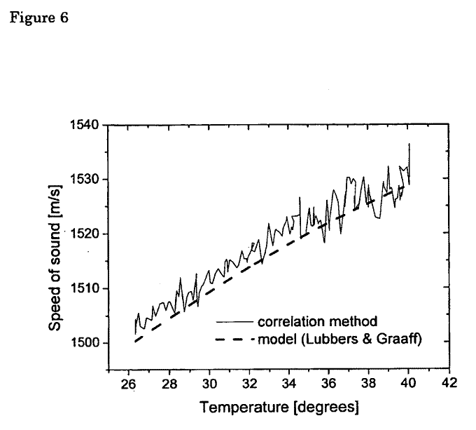

FIG. 6 shows the comparison of the acoustic velocity measurements with a reference model;

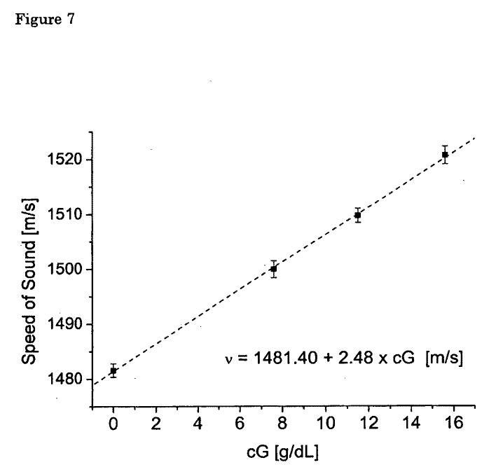

FIG. 7 shows a graph illustrating the measured speed of sound for different glucose concentration levels in water; and

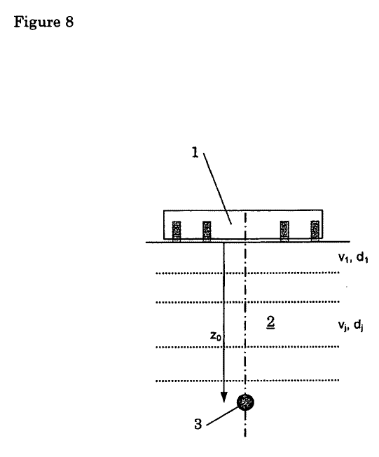

FIG. 8 shows a schematic view of determining a spatial distribution of velocity in tissue.

Turning to the figures, especially FIG. 1 and FIG. 2, according to the invention, a method is developed to online determine the speed of sound in tissue. In the illustrated preferred embodiment a photo acoustic sensor 1 is used coupling pulsed light into the tissue 2, for example, from a pulsed laser 4. The sensor works in reflection mode. The pulsed light induces acoustic waves in the tissue. Furthermore, the double ring structure of sensor 1 provides an extremely narrow aperture, so that the time traces can be regarded as a one-dimensional depth image of photo acoustic sources inside the measurement volume.

To generate photo acoustic signals, a pulsed light source 4 is used. In the embodiment a Q-switched Nd:YAG (Brilliant B, Quantel) pumping an optical parametric oscillator (Opotek) laser 4 was used, which generates light pulses of 710 nm with a duration of 10 ns, and a repetition rate of 10 Hz. The light is coupled into a glass fiber 5, which is integrated in the photo acoustic sensor 1. The sensor 1 comprises two concentric ring-shaped electrodes 6, 7. The inner ring 6 has an inner radius of 2 mm and a width of 0.17 mm. The outer ring 7 has an inner radius of 3.5 mm and a width of 0.1 mm, so that the area of both rings is equal. The electrodes are separated by a dielectric 8. The piezoelectric material 9 (25-micrometer-thick PVdF, biaxially stretched, electrically polarized, with one side metallized Au/Pt 10) is glued to the electrodes using significant pressure to minimize the thickness of the glue layer. The two ring shaped electrodes 6, 7 are connected to amplifiers (not shown). The sensor 1 is embedded in a brass housing 11 to shield the electronics for electromagnetic noise. An optical fiber 5 (core diameter 600 micrometer, NA 0.22) is placed in the center of the sensor 1 to deliver light pulses to the tissue. A schematic drawing of the cross section of the sensor is shown in FIG. 2. This sensor has an aperture of 3° (−6 dB of directivity function) for acoustic sources with a peak-to-peak time of 100 ns.

The inventive method is based on the fact that the signals detected by the two ring-shaped sensor areas 6, 7 arrive at different times. FIG. 3 shows the geometry of the receiver arrangement. The acoustic signals received from the receivers 6, 7 are inputted into computer 12. An oscilloscope 13 is used for timing the laser pulses and the recording of acoustic signals.

The computer 12 is programmed to perform a transformation on said plurality of acoustic signals to represent field values in a common computational point, based on a numerical estimate of the acoustic velocity in tissue 5 and receiver positions 3, 10 relative to a localized position of the acoustic source 3 (see FIG. 3). This transformation uses a numerical estimate of the acoustic velocity in tissue typically ranging from 1350 m/s for fat to 1700 m/s for skin. By applying a time delay with a numerical estimate of the acoustic velocity v₀ in the range, the time tᵢ′ at which the signals are detected by the rings is corrected such that the corrected time t corresponds to the depth z₀ (t=z₀/v₀) of the acoustic source: t={[(v₀ tᵢ′)²−Rᵢ²]^{1/2}}/v₀, where Rᵢ is the radius to the center of the inner ring 6 (in) or outer ring 7 (out) of the ring-shaped detection area and v₀ the speed of sound.

Assumption of a speed of sound v deviating from the real speed of sound v₀ will lead to not exactly coinciding signals P(t) of both rings 6, 7, after transformation. To estimate the coincidence of the signals a cross-correlation is calculated C(Δt)≡∫Pᵢₙ(t)Pₒᵤₜ(t+Δt)dt between the two signals. C(Δt) will be maximum for the time delay Δt that is needed to make the signals of the inner and outer ring exactly coincident.

Calculating C(Δt=0, v)=C₀(v) as a function of speed of sound will display a maximum at the assumed speed v equal to the real speed of sound v₀ in the tissue. An analytical expression for C₀(v) has been derived which is fitted to the measured C₀(v) curve to yield the actual velocity v₀.

At a distance r from the acoustic source, the laser-induced pressure transient can be described by (M. W. Sigrist and F. K. Kneubühl, J. Acoust. Soc. Am. 64, 1652 (1978):

$$P ( r , t ) = - {P}_{m ax} ( r ) \sqrt{e} \frac{t - τ}{1 / 2 {τ}_{pp}} exp [ - \frac{1}{2} {( \frac{t - τ}{1 / 2 {τ}_{pp}} )}^{2} ] , \qquad ( 1 )$$

with τ=z₀/v₀, and τₚₚ the peak to peak time (time difference between the occurrence of the positive and negative peak). This acoustic wave is detected by the i^{th} ring-shaped sensor at a time tᵢ′=τ. From this time tᵢ′, the depth zᵢ is calculated by assuming a speed of sound v:

$$\begin{matrix}{z}_{i} = \sqrt{{( {vt}_{i}^{′} )}^{2} - {R}_{i}^{2}} \\ = \sqrt{{( \frac{v}{{v}_{0}} )}^{2} ( {z}_{0}^{2} + {R}_{i}^{2} - {R}_{i}^{2} )} ,\end{matrix} \qquad ( 2 )$$

with i=in, out. The depth zᵢ will only be equal to the real depth z₀ if the assumed speed of sound v is equal to the actual speed v₀. Based on this depth zᵢ now an expression can be derived for the laser-induced pressure wave Pᵢ(zᵢ,t) corrected for the time-delay, i.e. the acoustic wave in the center of the ring shaped sensor surface, by taking τᵢ=zᵢ/vᵢ. Using Pᵢ(zᵢ,t), the cross-correlation at Δt=0 as a function of speed of sound C₀(v) can be calculated:

$$\begin{matrix}{C}_{0} ( v ) ∝ \frac{\sqrt{π}}{2 {τ}_{pp}} [ 2 - {( \frac{{τ}_{i n} - {τ}_{out}}{1 / 2 {τ}_{pp}} )}^{2} ] exp [ - 1 / 4 ( \frac{{τ}_{i n} - {τ}_{out}}{1 / 2 {τ}_{pp}} ) ] , & ( 3 ) \\ {τ}_{i} ( v ) = \frac{\sqrt{\frac{{v}^{2}}{{v}_{0}^{2}} ( {z}_{0}^{2} + {R}_{i}^{2} ) - {R}_{i}^{2}}}{v} . & ( 4 )\end{matrix}$$

When a second order Taylor series expansion of τᵢ(v) around v₀ is made and is substituted this in equation 3 one can write:

$${C}_{0} ( v ) ∝ \frac{\sqrt{π}}{2 {τ}_{pp}} [ 2 - {( \frac{v - {v}_{0}}{{v}_{0}^{2}} )}^{2} {( A - \frac{v - {v}_{0}}{{v}_{0}} B )}^{2} ] exp [ - 1 / 4 {( \frac{v - {v}_{0}}{{v}_{0}^{2}} )}^{2} {( A - \frac{v - {v}_{0}}{{v}_{0}} B )}^{2} ] \qquad ( 5 )$$

with A=(Rᵢₙ⁴−Rₒᵤₜ⁴)/(z₀³τₚₚ), and B=2(Rᵢₙ²−Rₒᵤₜ²)/(z₀τₚₚ). This expression can be fitted to the measured C₀(v) curves to yield v₀, without prior knowledge about τₚₚ or z₀, as these parameters can also be determined from the fit. Since the laser light is coupled into the tissue central respective to the concentric electrodes 6, 7 the source 3 is localized on the geometric central axis of the receivers 6, 7. Furthermore, the computer 12 may be used to generate enhanced acoustic images using the acoustic velocity determined according to the above described inventive method.

Next, FIG. 4 shows the calculated C₀(v) curves for a spherical photo-acoustic source with a peak-to-peak time of 40 ns, located at three different depths in a medium with v₀=1540 m/s, for Rᵢₙ=2.085 mm, and Rₒᵤₜ=3.55 mm. As expected, the maximum of these curves occurs at v₀. With increasing depth, the cross-correlation curve broadens. This is caused by the difference in time delay between the inner and outer ring that decreases with increasing depth. Hence, the method is especially advantageous for determining the speed of sound in a material at short range distances.

FIG. 5 shows a series of measured and fitted correlation functions for acoustic velocity measurements at different temperatures in water. Demineralised water was degassed by boiling it for about 10 minutes. The water was left to cool down to room temperature before the measurement was started. During the measurement the temperature of the water was slowly increased to above 40° C., by heating the reservoir by a laboratory heating plate. The temperature of the water was measured with a thermocouple.

A 200 μm diameter black horse tail hair was illuminated through a 100 μm diameter fiber, placed at about 1 mm from the hair, by light from an Nd:YAG laser. The horse tail functions as a transducer, absorbing the light and generating an acoustic wave from an acoustic point source, located at a depth of about 10 mm with respect to the sensor, generating a bipolar acoustic signal with a peak-to-peak time of 40 ns. The acoustic time traces, detected by the inner and outer rings of our PA sensor were measured at a position exactly above the point source and were averaged 64 times. The measurements were performed with the sensor described with reference to FIG. 1 and FIG. 2.

The cross-correlation of the measured signals of the inner and outer ring is calculated as a function of speed of sound. In FIG. 5 the resulting curves are shown for 3 different temperatures (26.4, 35.3, and 40.1° C.). The actual speed is determined by fitting equation 5 to the obtained cross-correlation curves. These values are indicated in the graphs together with the standard error and the correlation coefficient. The correlation with the fitted curves is high (R2>0.999), and the standard error in the estimated speed of sound is less than 0.01%.

Next FIG. 6 shows the speed of sound as a function of the measured temperature range at atmospheric pressure, together with a reference model (Lubbers & Graaff (J. Lubbers, and R. Graaff, Ultrasound Med. Biol. 24, 1065 (1998)), using their coefficients C2. Comparison of our data with their model shows an average systematic error of 0.1%. The standard deviation of the estimated speed is 0.1%. The accuracy that follows from a single temperature experiment (standard deviation 0.007%) indicates that the inventive method is able to estimate the sound speed with accuracy better than 0.1%.

FIG. 7 shows the results of measured speed of sound for different glucose concentration levels in water. The sound speed v is related to the density ρ and the bulk modulus B of a medium according to: B=ρv². Changes in glucose concentration affect both the bulk modulus and density and thus the speed of sound. It is shown that the invention can be used for determining analyte concentrations such as glucose derived from a measured speed of sound. Alternatively, the method could be used to analyze dissolved gas concentration such as dissolved oxygen in tissue.

FIG. 8 shows a schematic view of determining a spatial distribution of velocity in tissue. Here the inventive method is repeatedly used in a scanning way through the tissue where for different acoustic sources located at different positions an effective acoustic velocity of tissue between the acoustic source and the common computational point is measured. In this way a spatial distribution of effective acoustic velocities can be determined by generating a plurality of acoustic sources located at different distances from the receivers; and repeatedly determining an effective acoustic velocity between each of said plurality of acoustic sources and said common computational point. For instance, the layers d1, v1 represent a first layer in the tissue, wherein at a distance d1 an acoustic source is located. For N layers wherein dᵢ, vᵢ represent the thicknesses and sound velocities in the i^{th} layer then, the effective speed of sound v₀, determined from an acoustic source on a depth z₀ is given by:

$$\frac{1}{{v}_{0}} = \frac{1}{{z}_{0}} {\sum }_{j = 1}^{N} \frac{{d}_{j }}{{v}_{j}} \qquad ( 6 )$$

For every layer dᵢ, vᵢ can be derived from calculation (5), so that the distribution of sound velocities can be determined.

Although the invention is illustrated using a ring sensor and photo-acoustic ultrasound measurements, other sensor geometries are also feasible for applying the inventive concept, for example a linear array or other type of geometries. Further, instead of photo-acoustic ultrasound sources, other sources could be used, for example in pulse-echo experiments. Once absorbing structures (e.g. blood vessels) are identified, the sensor can be positioned above these structures and the actual speed of sound in the tissue can be determined from the calculated C₀(v) curves. The resulting speed of sound will avoid deformation artifacts in the resulting ultrasound images and will improve their resolution. This method is not limited to photo acoustic imaging, but can also be applied in pulse-echo ultrasound imaging using any detection geometry with at least two sensor elements at different distances to the acoustic source. Furthermore, the method can be used to monitor temperature-induced changes in speed of sound inside tissue.

1. A method of determining a parameter representing an acoustic property of a material, comprising:
actuating an acoustic source to generate an acoustic pressure wave in said material originating from a localized source position;
placing an acoustic sensor comprising a plurality of acoustic receivers at mutually differing distances from the acoustic source in acoustic contact with the material;
measuring upon actuation of said acoustic source a plurality of acoustic signals from said acoustic receivers arising from the actuation of the acoustic source;
transforming said plurality of acoustic signals to represent field values in a common computational point, based on a numerical estimate of acoustic velocity in the material and the acoustic receiver positions relative to a localized position of the acoustic source;
computing a signal representing a measure of overlap between said transformed plurality of acoustic signals as a function of said numerical estimate of said acoustic property parameter; and
deriving said acoustic property parameter from said measure of overlap signal.

2. A method according to claim 1, wherein determining said acoustic property parameter is based on maximizing the signal representing a measure of overlap between said transformed plurality of acoustic signals.

3. A method according to claim 2, wherein the signal representing a measure of overlap between said transformed plurality of acoustic signals is a cross correlation function between the transformed plurality of acoustic signals from said acoustic receivers, expressing a correlation measure relative to an estimated speed of sound.

4. A method according to claim 3, wherein said signal representing a measure of overlap is fitted to a cross correlation function C₀(v)
$${C}_{0} ( v ) ∝ \frac{\sqrt{π}}{2 {τ}_{pp}} [ 2 - {( \frac{v - {v}_{0}}{{v}_{0}^{2}} )}^{2} {( A - \frac{v - {v}_{0}}{{v}_{0}} B )}^{2} ] exp [ - 1 / 4 {( \frac{v - {v}_{0}}{{v}_{0}^{2}} )}^{2} {( A - \frac{v - {v}_{0}}{{v}_{0}} B )}^{2} ]$$
wherein A=(Rᵢₙ⁴−Rₒᵤₜ⁴)/(z₀³τₚₚ), and B=2 (Rᵢₙ²−Rₒᵤₜ²)/(z₀τₚₚ); Rᵢₙ, Rₒᵤₜ defining a ring geometry of inner and outer annular acoustic receivers; z₀ defining a depth of an acoustic source; xₚₚ defining a time difference between a positive and a negative peak in the acoustic wave; v₀ defining an effective speed of sound; and v defining an estimated speed of sound in the material.

5. A method according to claim 1, wherein said acoustic source is formed by absorption of light pulses.

6. A method according to claim 5, wherein an object is placed in the material absorbing the light pulses and forming an acoustic source.

7. A method according to claim 1, wherein said acoustic source is localized on an axis forming a geometric center of the acoustic receivers.

8. A method according to claim 1, wherein the acoustic property parameter represents an effective acoustic velocity of the material between the acoustic source and the common computational point.

9. A method according to claim 8 wherein a spatial distribution of effective acoustic velocities is determined by generating a plurality of acoustic pressure waves from a plurality of acoustic sources located at different distances from the acoustic receivers; and
repeatedly determining an effective acoustic velocity between each of said plurality of acoustic sources and said common computational point.

10. A method according to claim 1, wherein the determined acoustic property parameter is used for estimation of a variation of temperature distribution of tissue material.

11. A method according to claim 1, wherein the determined acoustic property parameter of tissue material is used for estimation of variation of tissue constituent concentrations.

12. A method according to claim 11, wherein said concentration is a glucose concentration.

13. A method according to claim 1, wherein the determined acoustic property parameter is used for estimation of dissolved gas concentration in the tissue material.

14. A system for-determining a parameter representing an acoustic property of a material, arranged for cooperating with an acoustic source actuator to generate an acoustic pressure wave in the material originating from a localized acoustic source position in the material and a sensor comprising a plurality of acoustic receivers to be placed in acoustic contact with the material at different positions thereon, for measuring, upon actuation of said acoustic source, a plurality of acoustic signals, said system comprising:
an input section for inputting said plurality of acoustic signals;
a computational unit for transforming said plurality of acoustic signals to represent field values in a common computational point, based on a numerical estimate of acoustic velocity in the material and the acoustic receiver positions relative to a localized position of the acoustic source; the computational unit further arranged for computing a signal representing a measure of overlap between said transformed plurality of acoustic signals as a function of said numerical estimate of said acoustic property parameter; and
an output section for outputting said acoustic property parameter derived from said measure of overlap signal.

15. A system according to claim 14, wherein said acoustic source is localized on an axis forming a geometric center of the acoustic receivers.

16. A system according to claim 14, wherein the sensor is a narrow aperture sensor.

17. A system according to claim 14, wherein the acoustic receivers in said sensor are arranged concentrically.

18. A system according to claim 17, wherein the acoustic receivers are annular in form.

19. A system according to claim 14, wherein said acoustic source actuator is a pulsed light source for inducing acoustic pressure waves in said material originating from a localized source position from absorption of said pulsed light.

20. A system according to claim 19, wherein the sensor comprises a light guide central to the receivers for guiding light of the pulsed light source into the material.

21. A system according to claim 14, wherein the system further comprises an image forming device, and wherein said acoustic property parameter is an acoustic velocity of an examined region in the material, for forming an image of said examined region based on said acoustic velocity.

22. A computer program product, comprising computer-executable program code portions for performing steps of:
actuating an acoustic source to generate an acoustic pressure wave in said material originating from a localized source position;
placing an acoustic sensor comprising a plurality of acoustic receivers at mutually differing distances from the acoustic source in acoustic contact with the material;
measuring upon actuation of said acoustic source a plurality of acoustic signals from said acoustic receivers arising from the actuation of the acoustic source;
transforming said plurality of acoustic signals to represent field values in a common computational point, based on a numerical estimate of acoustic velocity in the material and the acoustic receiver positions relative to a localized position of the acoustic source;
computing a signal representing a measure of overlap between said transformed plurality of acoustic signals as a function of said numerical estimate of said acoustic property parameter; and
deriving said acoustic property parameter from said measure of overlap signal.

23. An article of manufacture with a computer usable medium having computer readable instructions therein for providing access to resources available on that computer, the computer readable instructions, when executed by the computer, causing computer to perform the steps of:
actuating an acoustic source to generate an acoustic pressure wave in said material originating from a localized source position;
placing an acoustic sensor comprising a plurality of acoustic receivers at mutually differing distances from the acoustic source in acoustic contact with the material;
measuring upon actuation of said acoustic source a plurality of acoustic signals from said acoustic receivers arising from the actuation of the acoustic source;
transforming said plurality of acoustic signals to represent field values in a common computational point, based on a numerical estimate of acoustic velocity in the material and the acoustic receiver positions relative to a localized position of the acoustic source;
computing a signal representing a measure of overlap between said transformed plurality of acoustic signals as a function of said numerical estimate of said acoustic property parameter; and
deriving said acoustic property parameter from said measure of overlap signal.
